(19) United States
(12) Patent Application Publication
(10) Pub. No.: US 2025/0177739 A1
(43) Pub. Date: Jun. 5, 2025

(54) ATRAUMATIC RELEASE OF EPICARDIAL PACING WIRE

(71) Applicant: THE JOHNS HOPKINS UNIVERSITY, Baltimore, MD (US)

(72) Inventors: Glenn WHITMAN, Baltimore, MD (US); Youseph YAZDI, Ellicott City, MD (US)

(21) Appl. No.: 18/845,082

(22) PCT Filed: Feb. 2, 2023
(86) PCT No.: PCT/US2023/012193

§ 371 Date: Sep. 9, 2024

Related U.S. Application Data
(60) Provisional application No. 63/320,861, filed on Mar. 17, 2022.

(51) Int. Cl. A61N 1/05; A61N 1/362

(52) U.S. Cl. CPC A61N 1/059; A61N 1/362

(57) ABSTRACT

A system for pacing a heart of a patient includes a wire having a first end and a second end. The first end is configured to be connected to a pulse generator. The second end includes one or more connectors. The system also includes one or more anchors that are configured to be attached to the heart of the patient. The one or more connectors are configured to be releasably connected to the one or more anchors. The pulse generator is configured to transmit electrical pulses through the wire and the one or more connectors to the heart to pace the heart after a cardiac surgery.

### CROSS-REFERENCE TO RELATED APPLICATIONS

This application is the national stage entry of International Patent Application No. PCT/US2023/012193, filed on Feb. 2, 2023, and published as WO 2023/177483 A1 on Sep. 21, 2023, which claims the benefit of U.S. Provisional Patent Application No. 63/320,861, filed on Mar. 17, 2022, which are hereby incorporated by reference in their entireties.

### FIELD OF THE DISCLOSURE

The present disclosure relates generally to systems and methods for releasing a wire in a body. More particularly, the present disclosure relates to systems and methods for atraumatically releasing an epicardial pacing wire from the heart of a patient.

### BACKGROUND OF THE DISCLOSURE

One or more epicardial wire(s) may be attached to a patient's heart via sutures and/or clips during cardiac surgery. The wire(s) allow temporary pacing of the heart after the surgery to treat temporary rhythm disturbances or optimize cardiac performance. A few days after the surgery, the wire may be removed from the heart by exerting a pulling force on the wire that detaches the wire from the heart. Sometimes, this method of detaching the wire may damage the heart (e.g., cause bleeding). In rare instances, it may lead to the death of the patient. Therefore, what is needed is an improved system and method for atraumatically releasing an epicardial pacing wire from the heart of a patient.

### SUMMARY

A system for pacing a heart of a patient is described herein. The system includes a wire having a first end and a second end. The first end is configured to be connected to a pulse generator. The second end includes one or more connectors. The system also includes one or more anchors that are configured to be attached to the heart of the patient. The one or more connectors are configured to be releasably connected to the one or more anchors. The pulse generator is configured to transmit electrical pulses through the wire and the one or more connectors to the heart to pace the heart after a cardiac surgery.

In another embodiment, the system includes a pulse generator positioned outside of the patient. The system also includes an epicardial wire having a first end and a second end. The first end is configured to be connected to the pulse generator. The second end includes a plurality of leads. A length of each lead is from about 0.5 cm to about 5 cm. Each lead includes a connector. A cross-sectional width of each of each connector decreases proceeding toward the first end of the epicardial wire. The system also includes a plurality of anchors that are configured to be attached to the heart of the patient via one or more sutures, clips, hooks, corkscrews, glue, or a combination thereof. A distance between two adjacent anchors on the heart is from is from about 0.5 cm to about 5 cm. A surface area of each connector that faces a corresponding anchor is substantially flat and from about 1 mm² to about 2 cm². A surface area of each anchor that faces a corresponding connector is substantially flat and from about 5 mm² to about 3 cm². The pulse generator is configured to transmit electrical pulses through the epicardial wire, one or more of the connectors, one or more of the anchors, and to the heart to pace the heart after a cardiac surgery. The connectors, the anchors, or both include permanent magnets. The connectors are configured to be releasably connected to a corresponding one of the anchors via a magnetic force. The connectors are configured to be released from the anchors and withdrawn from the patient in response to a pulling force on the epicardial wire that exceeds the magnetic force.

A method for pacing a heart of a patient is also described. The method includes attaching one or more anchors to the heart. The method also includes connecting a first end of a wire to a pulse generator. The method also includes connecting a second end of the wire to the one or more anchors. The method also includes transmitting electrical pulses form a pulse generator, through the wire, to the heart to pace the heart after a surgery. The method also includes pulling the wire to cause the second end of the wire to release from the one or more anchors and be withdrawn from the patient.

### BRIEF DESCRIPTION OF THE FIGURES

FIG. 1 illustrates a schematic view of a system for pacing a heart of a patient, according to an embodiment.

FIG. 2 illustrates an enlarged view of a wire of the system (of FIG. 1) attached to the heart of the patient, according to an embodiment.

FIG. 3 illustrates a flowchart of a method for pacing the heart of the patient, according to an embodiment.

### DETAILED DESCRIPTION OF THE PREFERRED EMBODIMENTS

The presently disclosed subject matter now will be described more fully hereinafter with reference to the accompanying Drawings, in which some, but not all embodiments of the disclosures are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

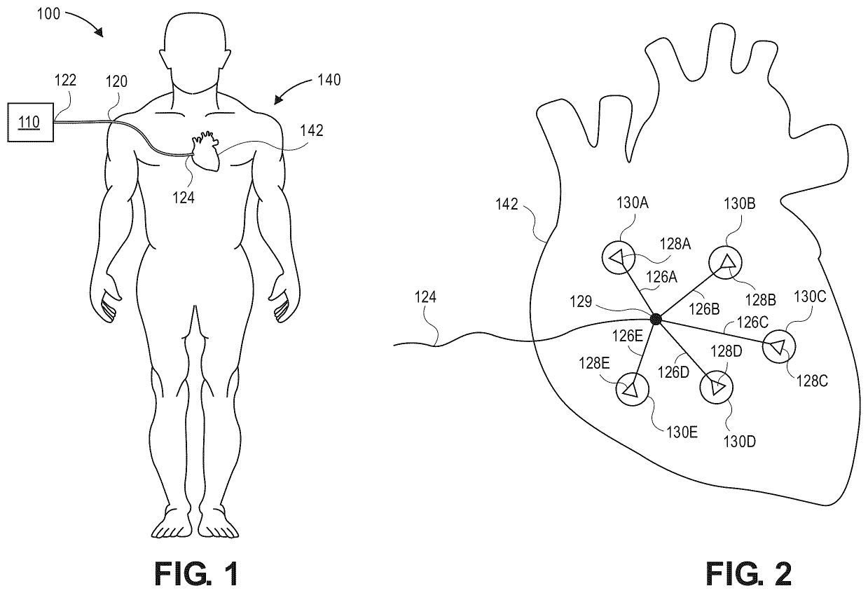

FIG. 1 illustrates a schematic view of a system 100 for pacing a heart 142 of a patient 140, according to an embodiment. The system 100 may include a pulse generator 110 and one or more wires (one is shown: 120). The wire 120 may be or include an epicardial wire. The wire 120 may include a first (e.g., external) end 122 a second (e.g., internal) end 124. The external end 122 is configured to be attached to the pulse generator 110, which is located outside of the patient 140. The internal end 124 is configured to be attached to the heart 142 of the patient 140 during surgery (e.g., cardiac surgery). Thus, the wire 120 may extend at least partially through the chest of the patient 140. As described in greater detail below, the pulse generator 110 may transmit electrical pulses through the wire 120 to the heart 142 to pace the heart 142 for one or more days (e.g., a week) after surgery.

FIG. 2 illustrates an enlarged view of the wire 120 attached to the heart 142 of the patient 140, according to an embodiment. In one embodiment, the wire 120 may be braided wire including one or more leads (five are shown: 126A-126E). As shown, the internal end 124 of the wire 120 may include the leads 126A-126E. Each lead 126A-126E may include one or more connectors 128A-128E, which may be configured to releasably connect to the heart 142.

The system 100 may also include one or more anchors (five are shown: 130A-130E) that may be attached to the heart 142 (e.g., the myocardium). The connectors 128A-128E may be configured to releasably connect to the anchors 130A-130E, thereby indirectly releasably connecting the wire 120 to the heart 142.

Having multiple leads 126A-126E, connectors 128A-128E, and/or anchors 130A-130E (i.e., multiple releasable connections) may cause the pulling force required to release the connections to be divided and/or spread out (i.e., not concentrated) on the heart 142. More particularly, the pulling force exerted on any single connector 128A-128E to cause that connector 128A-128E to release from the corresponding anchor 130A-130E may be less than a first threshold force that would cause damage to the heart 142 (e.g., cause tearing and/or bleeding). However, the pulling force on the wire 120 (e.g., exerted on two or more (e.g., all) of the connectors 128A-128E) to cause two or more (e.g., all) of the connectors 128A-128E to release from the corresponding anchors 130A-130E may be greater than a second threshold force to withstand inadvertent pulling so as to maintain at least one lead/connector/anchor releasable connection to transmit the electrical pulses to the heart 142.

Electrical pulses may be transmitted from the pulse generator 110, through the wire 120, to the heart 142 to pace the heart 142. More particularly, the pulses may be pass through one or more of the leads 126A-126E, one or more of the connectors 128A-128E, and/or one or more of the anchors 130A-130E. In one embodiment, a single lead 126C (e.g., having the farthest connector 128C from an intersection point 129 of the leads 126A-126E) may be configured to provide the pulse. This may ensure that the pulse is provided even if one or more of the other connections is inadvertently released. In another embodiment, each of the leads 126A-126E may be configured to provide the pulses (e.g., simultaneously) to the heart 142. This may also ensure that the pulse is provided even if one or more of the connections is inadvertently released.

A length of each lead 126A-126E may be from about 0.5 cm to about 5 cm or about 1 cm to about 3 cm. The length of each lead 126A-126E refers to the distance between the end of each lead 126A-126E (e.g., each connector 128A-128E) and the intersection point 129 (e.g., where two or more of the leads 126A-126 intersect). The lengths of the leads 126A-126E may be the same or different, as discussed below. The lengths of the leads 126A-126E may allow the connectors 128A-128E to be spread out from one another on the heart 142 so that the pulling force to release the connections and withdraw the wire 120 is spread out (i.e., not concentrated at one location) on the heart 142.

A surface area of each connector 128A-128E that faces and/or contacts a corresponding anchor 130A-130E may be substantially flat and from about 1 mm² to about 2 cm², about 2 mm² to about 1.5 cm², or about 5 mm² to about 1 cm². A cross-sectional width (e.g., diameter) of the connectors 128A-128E may remain constant and/or decrease proceeding toward the intersection point 129 on the wire 120. In the example shown, the connectors 128A-128E may have triangular or teardrop shapes, which may permit the connectors 128A-128E to be atraumatically pulled through the body after the connectors 128A-128E are released from the anchors 130A-130E and the wire 120 is withdrawn from the patient 140. In other embodiments, the connectors 128A-128E may have other shapes such as circles, rectangles (e.g., flattened cylinders), or the like.

The anchors 130A-130E may be attached to the heart 142 via sutures, clips, hooks, corkscrews, glue, or a combination thereof. In one embodiment, the anchors 130A-130E may be permanently attached to the heart 142 (e.g., remain attached until a subsequent surgery or the death of the patient 140). In another embodiment, the anchors 130A-130E may be configured to detach from the heart 142 over time. For example, the sutures may dissolve, and/or the anchors 130A-130E may be made of a biocompatible material that dissolves within the body over time (e.g., one month).

A distance between two adjacent anchors (e.g., anchors 130A, 130B) on the heart 142 may be from about 0.5 cm to about 5 cm or about 1 cm to about 3 cm. This may allow the connectors 128A-128E and/or the anchors 130A-130E to be spread out from one another on the heart 142 so that the pulling force to release the connections and withdraw the wire 120 is spread out (i.e., not concentrated at one location) on the heart 142.

A surface area of each anchor 130A-130E that faces and/or contacts a corresponding connector 128A-128E may be substantially flat and greater than the surface area of the connector 128A-128E. For example, the surface area may be from about 5 mm² to about 3 cm², about 10 mm² to about 2 cm², or about 20 mm² to about 1 cm². Although shown as having circular shapes, the anchors 130A-130E may also or instead have other shapes such as square, rectangular, ovular, etc. In one embodiment, two or more of the anchors 130A-130E may have different shapes.

In one embodiment, the anchors 130A-130E may be attached to the heart 142 such that, when the connectors 128A-128E are releasably connected to the anchors 130A-130E, a distance between each connector 128A-128E (and/or anchor 130A-130E) and the intersection point 129 is substantially the same (e.g., within about 5 mm or less). As a result, when a pulling force is exerted on the wire 120, the force is distributed substantially evenly across the connections. This may increase the force required to release the connections, which may help to prevent inadvertent releases.

In another embodiment, the anchors 130A-130E may be attached to the heart 142 such that, when the connectors 128A-128E are releasably connected to the anchors 130A-130E, a distance between each connector 128A-128E (and/or anchor 130A-130E) and the intersection point 129 is different (e.g., staggered). For example, the distance between the connector 128A (and/or anchor 130A) and the intersection point 129 may be less than the distance between the connector 128B (and/or anchor 130B) and the intersection point 129. As a result, the connection between the connector 128A and the anchor 130A may be released prior to the connection between the connector 128B and the anchor 130B. This may reduce the force required to release each connection, which may reduce the force exerted on the heart 142. In addition, it may provide one or more fallback connections in the event that one of the connections (e.g., between the connector 128A and the anchor 130A) is inadvertently released.

In one embodiment, the connectors 128A-128E may be configured to releasably connect to the anchors 130A-130E via magnetism. More particularly, the connectors 128A-128E and/or the anchors 130A-130E may be made from a ferromagnetic and/or ferrimagnetic material such that they may be attracted to one another via magnetic force. In one example, the connectors 128A-128E and/or the anchors 130A-130E may be or include permanent magnets, and the connection therebetween may be released in response to exerting a pulling force on the wire 120 that is greater than the magnetic attraction force. In another example, the connectors 128A-128E may be or include electromagnets, and the connection between the connectors 128A-128E and the anchors 130A-130E may be released by turning off the electrical power supplied to the connectors 128A-128E that generates the electromagnetic force and then pulling on the wire 120.

In another embodiment, the connectors 128A-128E may be configured to releasably connect to the anchors 130A-130E via hooks and loops (e.g., VELCRO®). For example, the connectors 128A-128E may include one or more hooks, and the anchors 130A-130E may include one or more loops, or vice versa. The strength of the connections (and/or the force required to release the connections) may be tuned by varying the number of hooks and loops that are selected.

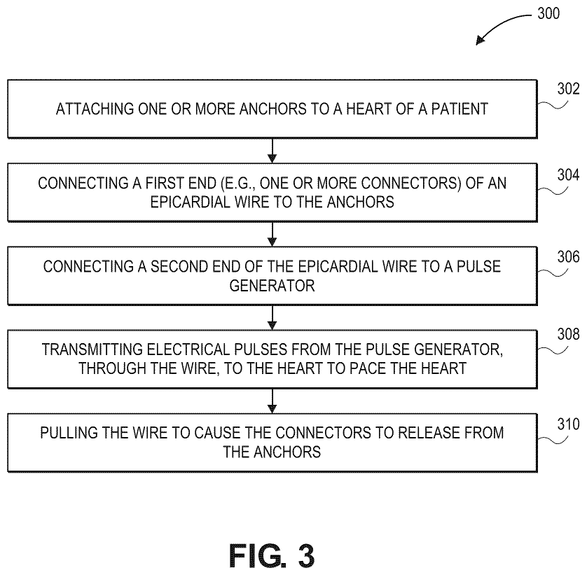

FIG. 3 illustrates a flowchart of a method 300 for pacing the heart 142 of the patient 140, according to an embodiment. An illustrative order of the method 300 is described below; however, one or more steps of the method 300 may be performed in a different order, combined, split into sub-steps, repeated, or omitted.

The method 300 may include attaching the anchor(s) 130A-130E to the heart 142 of the patient, as at 302. The anchors 130A-130E may be attached during a (e.g., cardiac) surgery using sutures, clips, etc.

The method 300 may also include connecting the end 124 of the wire 120 to the anchors 130A-130E, as at 304. More particularly, the connectors 128A-128E may be releasably connected to the anchors 130A-130E via magnetism, hooks and loops, etc. during the surgery. The wire 120 may extend through the chest of the patient 140 after the surgery is concluded.

The method 300 may also include connecting the end 122 of the wire 120 to the pulse generator 110, as at 306.

The method 300 may also include transmitting electrical pulses from the pulse generator 110, through the wire 120, to the heart 142, as at 308. The pulses may pass through one or more of the leads 126A-126E, one or more of the connectors 128A-128E, and/or one or more of the anchors 130A-130E. The pulses may pace the heart 142.

After a few days, when the wire 120 is ready to be removed, the method 300 may also include pulling the wire 120 to cause the connectors 128A-128E to release from the anchors 130A-130E, as at 310. The wire 120 (e.g., the leads 126A-126E and/or connectors 128A-128E) may then be withdrawn from the body via the chest. The anchors 130A-130E may remain attached to the heart 142.

As used herein, the terms “inner” and “outer”; “up” and “down”; “upper” and “lower”; “upward” and “downward”; “upstream” and “downstream”; “above” and “below”; “inward” and “outward”; and other like terms as used herein refer to relative positions to one another and are not intended to denote a particular direction or spatial orientation. The terms “couple,” “coupled,” “connect,” “connection,” “connected,” “in connection with,” and “connecting” refer to “in direct connection with” or “in connection with via one or more intermediate elements or members.”

Although the present disclosure has been described in connection with preferred embodiments thereof, it will be appreciated by those skilled in the art that additions, deletions, modifications, and substitutions not specifically described may be made without departing from the spirit and scope of the disclosure as defined in the appended claims.

1. A system for pacing a heart of a patient, the system comprising:
a wire having a first end and a second end, wherein the first end is configured to be connected to a pulse generator, and wherein the second end comprises one or more connectors; and
one or more anchors that are configured to be attached to the heart of the patient, wherein the one or more connectors are configured to be releasably connected to the one or more anchors, and wherein the pulse generator is configured to transmit electrical pulses through the wire and the one or more connectors to the heart to pace the heart after a cardiac surgery.

2. The system of claim 1, wherein a cross-sectional width of each connector decreases proceeding toward the first end of the wire.

3. The system of claim 1, wherein the second end of the wire comprises a plurality of leads, and wherein each of the leads comprises one of the one or more connectors.

4. The system of claim 3, wherein a length of each lead is from about 0.5 cm to about 5 cm.

5. The system of claim 1, wherein the one or more anchors are attached to the heart via one or more sutures, clips, hooks, corkscrews, glue, or a combination thereof.

6. The system of claim 1, wherein a surface area of the one or more connectors that faces the one or more anchors is less than a surface area of the one or more anchors that faces the one or more connectors.

7. The system of claim 1, wherein the one or more connectors, the one or more anchors, or both comprise permanent magnets that releasably connect the one or more connectors to the one or more anchors.

8. The system of claim 1, wherein the one or more connectors and the one or more anchors comprise hooks, loops, or both that releasably connect the one or more connectors to the one or more anchors.

9. The system of claim 1, wherein the one or more connectors are configured to be released from the one or more anchors and withdrawn from the patient in response to a pulling force on the wire.

10. The system of claim 9, wherein the one or more anchors are configured to remain attached to the heart after the wire is withdrawn from the patient.

11. A system for pacing a heart of a patient, the system comprising:
a pulse generator positioned outside of the patient;
an epicardial wire having a first end and a second end, wherein the first end is configured to be connected to the pulse generator, wherein the second end comprises a plurality of leads, wherein a length of each lead is from about 0.5 cm to about 5 cm, wherein each lead comprises a connector, and wherein a cross-sectional width of each of each connector decreases proceeding toward the first end of the epicardial wire; and
a plurality of anchors that are configured to be attached to the heart of the patient via one or more sutures, clips, hooks, corkscrews, glue, or a combination thereof, wherein a distance between two adjacent anchors on the heart is from is from about 0.5 cm to about 5 cm, wherein a surface area of each connector that faces a corresponding anchor is substantially flat and from about 1 mm² to about 2 cm², and wherein a surface area of each anchor that faces a corresponding connector is substantially flat and from about 5 mm² to about 3 cm²,
wherein the pulse generator is configured to transmit electrical pulses through the epicardial wire, one or more of the connectors, one or more of the anchors, and to the heart to pace the heart after a cardiac surgery, wherein the connectors, the anchors, or both comprise permanent magnets, wherein the connectors are configured to be releasably connected to a corresponding one of the anchors via a magnetic force, and wherein the connectors are configured to be released from the anchors and withdrawn from the patient in response to a pulling force on the epicardial wire that exceeds the magnetic force.

12. The system of claim 11, wherein the lengths of the leads are substantially the same so that the pulling force is distributed to each of the connectors simultaneously.

13. The system of claim 11, wherein the lengths of the leads are different so that the pulling force is configured to be exerted on a first of the connectors without being exerted on a second of the connectors.

14. The system of claim 11, wherein a first of the leads is longer than a remainder of the leads so that the pulling force is configured to be exerted on the remainder of the leads without being exerted on the first lead, wherein the first lead transmits the electrical pulses, and wherein the remainder of the leads do not transmit the electrical pulses.

15. The system of claim 11, wherein the pulling force exerted on a first of the connectors to cause the first connector to release from a first of the anchors is less than a first threshold force that would cause damage to the heart, and wherein the pulling force exerted on the epicardial wire to cause all of the connectors to release from all of the anchors is greater than a second threshold force.

16. A method for pacing a heart of a patient, the method comprising:
attaching one or more anchors to the heart;
connecting a first end of a wire to a pulse generator;
connecting a second end of the wire to the one or more anchors;
transmitting electrical pulses form a pulse generator, through the wire, to the heart to pace the heart after a surgery; and
pulling the wire to cause the second end of the wire to release from the one or more anchors and be withdrawn from the patient.

17. The method of claim 16, wherein attaching the one or more anchors comprises attaching a first anchor and a second anchor to the heart, wherein a distance between the first and second anchors on the heart is from about 0.5 cm to about 5 cm.

18. The method of claim 17, wherein the second end of the wire comprises:
a first lead having a first connector; and
a second lead having a second connector,
wherein connecting the second end of the wire comprises:
releasably connecting the first connector to the first anchor via magnetism or hooks and loops; and
releasably connecting the second connector to the second anchor via magnetism or hooks and loops.

19. The method of claim 18, wherein pulling the wire exerts simultaneous forces on the first and second connectors.

20. The method of claim 18, wherein pulling the wire does not exert a force on the second connector until the first connector releases from the first anchor.
